(19) United States
(12) Patent Application Publication
(10) Pub. No.: US 2021/0307808 A1
(43) Pub. Date: Oct. 7, 2021

(54) METHOD AND SYSTEM FOR SUPPORTING AN HF SURGICAL PROCEDURE AND SOFTWARE PROGRAM PRODUCT

(71) Applicant: OLYMPUS WINTER & IBE GMBH, Hamburg (DE)

(72) Inventor: Jens KRÜGER, Eichwalde (DE)

(73) Assignee: OLYMPUS WINTER & IBE GMBH, Hamburg (DE)

(21) Appl. No.: 17/191,957

(22) Filed: Mar. 4, 2021

(30) Foreign Application Priority Data
Mar. 4, 2020 (DE) 10 2020 105 835.7

(51) Int. Cl. A61B 18/14; A61B 5/00; A61B 1/05; A61B 1/00; A61B 18/12

(52) U.S. Cl. CPC A61B 18/14; A61B 5/0075; A61B 5/7267; A61B 2018/00589; A61B 1/00013; A61B 18/1206; A61B 1/05

(57) ABSTRACT

A method and system for supporting an HF surgical procedure in which tissue is treated. The method includes supplying an HF instrument including an HF electrode and an HF generator with HF current, providing a plurality of HF modes adapted to respective ones of a plurality of tissue types, and orienting an optical capturing device toward the HF electrode such that a field of view of the optical capturing device is configured to encompass a region of the tissue to be treated around the HF electrode during an intended treatment of the tissue. The method further includes performing an optical classification of a tissue type of the tissue in the region of the HF electrode based on optical measurement signals captured by the optical capturing device, and setting a specific HF mode for the tissue type based on the result of the optical classification.

### BACKGROUND

This application relates to a method for supporting an HF (high frequency) surgical procedure in which tissue is treated, for example cut or coagulated, with an endoscopic HF instrument. For various tissue types, various HF modes adapted to the tissue types are available, as well as a software program product.

An HF surgical system is sold by the applicant under the name ESG, which comprises a series of HF generators. Tissue can be cut and coagulated, among other things, with monopolar or bipolar HF instruments. Depending on the tissue that must be processed with the ESG generator system, for example fat tissue, muscle tissue, connective tissue, etc., the cutting or coagulating by means of HF (high frequency) current achieves different results. Suitably adapted HF modes which lead to good treatment results are available for most tissue types. An HF mode generated by the HF generator should, in the ideal case, be able to adapt itself to these situations and, depending on the tissue, bring the thermal output into the tissue in different ways, especially corresponding to the known HF modes.

This automatic adaptation has, until now, only been possible to a limited extent. Until now, the tissue to be operated on has been characterized by its electrical properties, namely impedance and resistance in the mode. However, these purely electrical properties of the tissue are not sufficient to reliably detect the tissue type. As a result, it is not possible with the existing HF modes to change the behavior of the HF mode when transitioning between different tissue types, for example when transitioning from muscle tissue to fat tissue with different resistance. Using existing HF modes, this type of action leads to the cutting or coagulating at a transition from one tissue type to another tissue type with different electrical properties taking place with the previously set HF mode which is not ideal for the new tissue type. That is, neighboring tissue, for example nerve pathways or blood vessels, can be unintentionally cut or coagulated.

### SUMMARY

In contrast, an object of the present application is to provide a method and system for supporting an HF surgical procedure with which the treatment result is improved.

This object is achieved by a method for supporting an HF surgical procedure in which tissue is treated. The method includes supplying an HF instrument including an HF electrode and an HF generator with HF current, providing a plurality of HF modes adapted to respective ones of a plurality of tissue types, and orienting an optical capturing device toward the HF electrode such that a field of view of the optical capturing device is configured to encompass a region of the tissue to be treated around the HF electrode during an intended treatment of the tissue.

The method further includes performing an optical classification of a tissue type of the tissue in the region of the HF electrode based on optical measurement signals captured by the optical capturing device, and setting a specific HF mode for the tissue type based on the result of the optical classification. This application is based on the fundamental concept that, by using an optical analysis and classification of the tissue to be treated, a reliable detection of the tissue type or tissue types lying in the field of view of the optical capturing device is possible, such that at the transition of the treatment from one tissue type to another tissue type, a reliable basis is present for adapting the HF mode to the new tissue type and thus for preventing improper treatment. The optical analysis requires the integration of an optical sensor system in or on the endoscopic HF instrument and an analysis of the signals generated by the optical sensor system with regard to the classification of the tissue type.

In practice, there are many methods for characterizing abiotic and biotic surfaces and materials, but these have not yet been used in the context of HF surgery until now. This includes simple imaging with a camera system, a flat image sensor, for example CCD (charge-coupled device) or CMOS (complementary metal-oxide semiconductor), with corresponding signal processing, or also optical spectroscopy methods. If, depending on the method, one or more optical waveguides are integrated, for example, toward the instrument, these methods can be used to detect tissue types. An optical measuring head of an optical capturing device attached or integrated on the HF instrument then converts, for example, the optical information of the tissue into a transmittable format, for example interference patterns, via, for example, optical waveguides. Alternatively, the optical measuring data can also be transmitted electrically with suitable shielding. In addition to the tissue properties, the changes before and after the procedure can be evaluated and used, for example, for an automation of HF modes, for example to detect the coagulation result and to warn of insufficient hemostasis.

Advantageously, the classification of the tissue types includes a classification by the type and/or by properties of tissue types, for example regarding the conductivity. The classification of the tissue types is accompanied by HF modes for the corresponding tissue types, for example in “fat tissue,” “muscle tissue,” “liver” and the like, while the classification with regard to the properties of tissue types, for example regarding the conductivity, allows a structuring of the HF modes that correlate more directly with the physical properties of the tissue to be treated, for example “conductive” or “non-conductive.” The conductivity of tissue is an indicator of how well the output can be introduced into the tissue.

The tissue type detection has several advantages. For some tissue types, it must be ensured that the tissue does not dry out due to the output. For this purpose, HF modes are pulsed so that water can flow back into the tissue. The tissue detection allows the pulsing to be activated or modulated depending on the water content. When cutting occurs on the boundary between various tissue types, a control of the output can prevent too much tissue being cut or coagulated. When cutting tissue, a phase with high voltage or power output is usually connected in advance, which facilitates the cut. This preceding power output can also be dosed depending on the tissue. A preselection of modes suitable for the tissue type from the plurality of provided HF modes can also be made for a physician by means of the tissue detection.

The tissue type detection can also be used to detect a successful or an incomplete hemostasis, in which cases, for example, the introduction of the output automatically stops or a notification occurs. In addition, carbonization can be detected and prevented by reducing the output.

In embodiments of the method, the optical classification of the tissue type takes place based on a spectroscopic analysis, for example reflection spectroscopy, autofluorescence spectroscopy or Raman spectroscopy. The principle of Raman spectroscopy of biological tissue has been described, for example, in Z. Movasaghi et al., “Raman Spectroscopy of Biological Tissues”, Appl. Spectr. Rev., 42, 493-541 (2007). In addition, realtime skin analysis by means of Raman spectroscopy has been reported in J. Zhao et al., “Real-Time Raman Spectroscopy for Noninvasive in vivo Skin Analysis and Diagnosis.” Furthermore, the use of reflection spectroscopy and autofluorescence spectroscopy in tissue typing in the context of laser surgery was described in the doctoral dissertation of A. Zam, “Optical Tissue Differentiation for Sensor-Controlled Tissue-Specific Laser Surgery,” Erlangen (2011). In order to realize spectroscopic tissue typing in the context of endoscopic HF surgery, it is thus possible to first build a database with the spectral properties of the various tissue types to be examined or to be treated as a basis of comparison and to design the system such that the spectroscopic data obtained during the operation from the optical capturing device are compared with the corresponding spectroscopic data from the comparison tissue types.

Alternatively or additionally, in embodiments the optical classification of the tissue type takes place based on an analysis of color, shape and/or texture of the tissue with broadband visible light or narrow-band light in one narrow band or multiple narrow bands. In this case, it is an analysis of the optical data from an imaging method, which can be analyzed inter alia with regard to the colors, but also with regard to other properties such as typical patterns or shapes in the image that correspond with the shape or texture of the tissue. If narrow-band light is used, in order to support or enable the classification of the tissue, it is possible to cause characteristic structures of certain tissue types to become particularly clear through a short-term illumination with a light of a defined color.

In embodiments, the optical classification of the tissue type takes place using a neural network trained on the basis of comparable images, characteristic values from imaging methods, spectrograms and/or characteristic spectrographic data for the various tissue types or on the basis of a comparison with predetermined comparative values. If a learning system is used, it is possible to further train the learning system, for example the neural network, on the basis of data from actual HF surgical procedures, for example by comparing the result of the classification of the tissue with the electrical properties of the tissue measured by the generator, wherein in the case of a discrepancy between the two measurements, the learning system is notified that the classification is unreliable. On the basis of the measured electrical properties of the tissue, a hypothesis about which tissue was actually present can be formed and compared with the optical properties of various tissue types known from the comparative data.

In embodiments of the method, the optical measurement signals are evaluated with regard to whether tissue of another tissue type than that of the current tissue type located in the region of the HF electrode is present in the surroundings of the HF electrode, wherein in particular a distance of at least one region with the different tissue type from the current tissue type is monitored and a change in the HF mode to the HF mode appropriate for the different tissue type is initiated when the HF electrode reaches the region of the tissue with the different tissue type. This development relates to the use of the method during a procedure and means that the further surroundings of the current position of the HF electrode are examined by means of the optical capturing device and it is determined whether tissue with a different tissue type than the directly treated tissue type is present in these surroundings. In doing so, it is monitored whether the HF electrode approaches this other tissue type so that the HF generator is enabled to set another, more suitable HF mode when the tissue of the other tissue type is reached. With imaging methods as the basis of the classification, this is possible in that the edge regions of the image are examined in the same way as the region around the HF electrode. In spectroscopic examinations, either a flat measurement is also taken; alternatively, measurements can be taken at various points around the HF electrode, for example via spatially distributed optical waveguides, and each be evaluated individually.

Additionally, changes in the HF mode may be evaluated after an operation and used to improve and/or automate the HF modes and/or to improve the classification of the tissue types. With this measure, it is possible to improve the method on the basis of the measurements in actual use, and potentially both with regard to the reliable detection of the tissue types and also the improvement of the HF modes and if appropriate the creation of new HF modes that can be better adapted to the specific tissue regions than the existing HF modes. As long as the generator is technically able to collect operation data, the tissue properties that were changed by the HF modes can be statistically evaluated. As a result, the behavior of the HF modes on the tissue can be better characterized and corresponding statistical tissue models and better HF modes can be developed while at the same time reducing the number of tests on animals.

In embodiments, a result of the HF treatment, for example a coagulation result, is detected from the optical recordings underlying the optical classification of tissue types, wherein in the case of an insufficient result, for example an insufficient hemostasis, a warning is emitted.

An object of the application is also achieved by a system for supporting an HF surgical procedure in which tissue is treated. The system may include an HF instrument including an HF electrode and an HF generator, the HF generator configured to supply the HF instrument with HF current. The HF generator is configured to provide a plurality of HF modes adapted to respective ones of a plurality of tissue types.

The system may further include an optical capturing device, the optical capturing device provided as part of the HF instrument or connected thereto. The optical capturing device is oriented toward the HF electrode such that a field of view of the optical capturing device is configured to encompass a region of the tissue to be treated around the HF electrode during an intended treatment of the tissue.

The system may further include an evaluation device configured to (a) perform an optical classification of a tissue type of the tissue in the region of the HF electrode based on optical measurement signals captured by the optical capturing device, and to (b) set a specific HF mode for the tissue type based on the result of the optical classification.

The characteristics, features and advantages of the system according to the application correspond to those of the method according to the application.

In embodiments, the evaluation device is configured in the HF generator.

Advantageously, the evaluation device is configured to perform a previously described method according to the invention.

In embodiments, the optical capturing device comprises an optical waveguide or a bundle of optical waveguides that are integrated into the endoscopic HF instrument or can be fastened to the endoscopic HF instrument from the outside. For example, in the latter case, the optical capturing device is equipped with a clip with which it can be placed onto the endoscopis HF instrument. A signal line of the optical capturing device may also be connected to the cable via a fastening device, which cable connects the HF generator to the HF instrument for supply. In this manner, the optical capturing device is configured as a retrofittable auxiliary device or addition to the existing HF surgical system.

In embodiments, the optical capturing device comprises an imaging sensor and/or a spectrometer, in particular a reflection spectrometer, an autofluorescence spectrometer or a Raman spectrometer.

An object of the application is also achieved by a non-transitory, computer-readable medium that stores a program for causing a computer to execute performing an optical classification of a tissue type of a tissue in a region of an HF electrode based on optical measurement signals captured by an optical capturing device, and setting a specific HF mode for the tissue type based on the result of the optical classification. The non-transitory, computer readable medium thus realizes the features, advantages and characteristics of the previously described method according to the application and supplements the method and the system of the present application.

Further features of the application will become apparent from the description of embodiments according to the application together with the claims and the included drawing. Embodiments according to the application can fulfill individual features or a combination of several features.

In the scope of the invention, features which are designated by “for example” are understood to be optional features.

### BRIEF DESCRIPTION OF THE DRAWING

The invention is described below, without restricting the general idea of the invention, based on exemplary embodiments in reference to the drawing, whereby we expressly refer to the drawing with regard to the disclosure of all details according to the invention that are not explained in greater detail in the text. The figure shows:

FIG. 1 a schematic representation of a system according to the invention.

### DETAILED DESCRIPTION

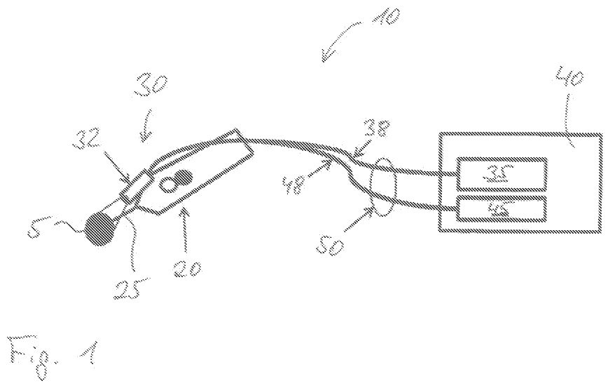

FIG. 1 shows a schematic representation of a system 10 according to the invention for supporting an HF surgical procedure during such a procedure. The system 10 comprises an endoscopic HF instrument 20 with a longitudinally extending endoscope shaft, at the distal tip of which an HF electrode 25 is arranged which is brought into contact with tissue 5 in order to heat it strongly in a localized manner by introducing HF output and as a result to cut or to coagulate. The HF electrode 25 can be a monopolar or a bipolar electrode.

The HF instrument 20 is connected to an HF generator 40 by an HF cable 48 which supplies the distal HF electrode 25 with HF current. The HF generator has HF measuring instrumentation 45 which is configured to measure electrical properties of the treated tissue 5, for example its electrical conductivity. The electrical conductivity determined by the HF measuring instrumentation 45 or other electrical properties determined by the HF measuring instrumentation 45 are used by the HF generator 40 to set suitable HF modes for the detected tissue type so that the optimal type of output for the tissue type can be introduced. This ensures that optimal treatment results for the respective tissue type are achieved.

The system 10 according to this embodiment also comprises an optical capturing device 30 which comprises an optical measuring head 32 and optical measuring instrumentation 35 which are connected to each other by optical waveguides 38, wherein the optical measuring instrumentation is arranged in the HF generator 40 in the exemplary embodiment. In the context of endoscopic HF surgery, optical waveguides as signal transmitters have the advantage that they are not negatively impacted by the HF fields generated by the HF electrode. If the cable is suitably shielded, however, it is also possible to realize electrical signal transmission for the data from the optical measuring head 32 to the optical measuring instrumentation 35.

The measuring head 32 can be configured completely optically without electrical components, for example through one or more optical waveguides 38 which are oriented toward the tissue around the HF electrode 25, if appropriate with an imaging optical system placed before it. Multiple optical waveguides 38 can also be led together to the distal tip of the endoscope shaft and distally spread toward the tissue such that each optical waveguide has a different small region of the tissue in its field of view, wherein the light that reaches the optical measuring instrumentation 35 through the various optical waveguides 38 is analyzed separately from each other. In this manner, classifications for the tissue types are present both at the location of the HF electrode 25 and also at various points around the HF electrode 25. Since the HF electrode 25 typically does not pause at one point of the tissue 5 during a treatment but is moved through the tissue or over the tissue, it is possible in this manner to detect the change in a tissue type along the direction of movement of the HF electrode 25 at an early stage and set a suitable other HF mode when this new tissue type is reached.

The optical measuring head 32 can be integrated into the HF instrument 20, but can also be configured as a retrofit solution and, as shown in FIG. 1, be fastened from the outside to the HF instrument in a suitable manner. This can be realized either through gripping means on the optical measuring head 32, or through an arrangement of the HF instrument 20 with an accommodation for an optical measuring head 32, wherein the optical measuring head 32 is configured with corresponding complementary means for the accommodation on the HF instrument 20. This solution enables the HF instrument to be equipped with various optical capturing devices which are optimized for various areas of application and purposes and may realize various optical measuring methods.

In the case of a retrofit solution, in order to prevent hindrances to the surgical personnel, one further development provides leading the various cables that lead to the HF measuring instrumentation 45 on the one hand and to the optical measuring instrumentation 35 on the other hand as a bundle 50. This can be done either through a common cable guide or cable integration, i.e. through a common cable for the HF and optical components, or through a mechanical bundling of the separate HF and optical cables or optical waveguides by means of a cable tunnel, by means of cable clamps, or the like.

All named characteristics, including those taken from the drawing alone, and individual characteristics, which are disclosed in combination with other characteristics, are considered alone and in combination as essential for the invention. Embodiments according to the invention can be fulfilled by individual features or a combination of several features.

### LIST OF REFERENCE SIGNS

5 Tissue

10 System

20 HF instrument

25 HF electrode

30 Optical capturing device

32 Optical measuring head

35 Optical measuring instrumentation

38 Optical waveguide

40 HF generator

45 HF measuring instrumentation

48 HF cable

50 Bundle

What is claimed is:

1. A method for supporting an HF surgical procedure in which tissue is treated, the method comprising:
supplying an HF instrument including an HF electrode and an HF generator with HF current;
providing a plurality of HF modes adapted to respective ones of a plurality of tissue types;
orienting an optical capturing device toward the HF electrode such that a field of view of the optical capturing device is configured to encompass a region of the tissue to be treated around the HF electrode during an intended treatment of the tissue;
performing an optical classification of a tissue type of the tissue in the region of the HF electrode based on optical measurement signals captured by the optical capturing device; and
setting a specific HF mode for the tissue type based on the result of the optical classification.

2. The method according to claim 1, wherein the optical classification of the plurality of tissue types is performed on the basis of type and/or by properties of tissue types.

3. The method according to claim 2, wherein the optical classification of the plurality of tissue types is performed on the basis of conductivity of the plurality of tissue types.

4. The method according to claim 1, wherein the optical classification of the plurality of tissue types is performed using a spectroscopic analysis.

5. The method according to claim 4, wherein the optical classification of the plurality of tissue types is performed using reflection spectroscopy, autofluorescence spectroscopy or Raman spectroscopy.

6. The method according to claim 1, wherein the optical classification of the plurality of tissue types is performed on the basis of an analysis of color, shape and/or texture of the tissue with broadband visible light or narrow-band light in one narrow band or multiple narrow bands.

7. The method according to claim 1, wherein the optical classification of the plurality of tissue types is performed using a neural network trained on the basis of comparable images, characteristic values from imaging methods, spectrograms and/or characteristic spectrographic data for the various tissue types or on the basis of a comparison with predetermined comparative values.

8. The method according to claim 1, wherein the optical measurement signals are evaluated with regard to whether tissue of another tissue type than that of a current tissue type located in the region of the HF electrode is present in the surroundings of the HF electrode, wherein a distance of at least one region with the different tissue type from the current tissue type is monitored and a change in the HF mode to an HF mode appropriate for the different tissue type is initiated when the HF electrode reaches the region of the tissue with the different tissue type.

9. The method according to claim 1, further comprising evaluating changes in the HF mode after an operation, the evaluation being used to improve and/or automate the HF modes and/or to improve the classification of the tissue types.

10. The method according to claim 1, further comprising detecting a result of the HF treatment from optical recordings underlying the optical classification of tissue types, wherein in the case of an insufficient result, a warning is emitted.

11. The method according to claim 10, wherein the result is a coagulation result.

12. The method according to claim 10, wherein the insufficient result is an insufficient homeostasis.

13. A system for supporting an HF surgical procedure in which tissue is treated, comprising:
an HF instrument including an HF electrode and an HF generator, the HF generator configured to supply the HF instrument with HF current,
wherein the HF generator is configured to provide a plurality of HF modes adapted to respective ones of a plurality of tissue types;
an optical capturing device, the optical capturing device provided as part of the HF instrument or connected thereto,
wherein the optical capturing device is oriented toward the HF electrode such that a field of view of the optical capturing device is configured to encompass a region of the tissue to be treated around the HF electrode during an intended treatment of the tissue; and
an evaluation device configured to (a) perform an optical classification of a tissue type of the tissue in the region of the HF electrode based on optical measurement signals captured by the optical capturing device, and to (b) set a specific HF mode for the tissue type based on the result of the optical classification.

14. The system according to claim 13, wherein the evaluation device is provided in the HF generator.

15. The system according to claim 13, wherein the optical capturing device comprises an optical waveguide or a bundle of optical waveguides that are integrated into the endoscopic HF instrument or can be fastened to the endoscopic HF instrument from outside.

16. The system according to claim 13, wherein the optical capturing device comprises an imaging sensor.

17. The system according to claim 13, wherein the optical capturing device comprises a spectrometer.

18. The system according to claim 13, wherein the optical capturing device includes a measuring head including the optical waveguide or the bundle of optical waveguides, the measuring head being constructed without electrical components.

19. A non-transitory, computer-readable medium that stores a program for causing a computer to execute:
performing an optical classification of a tissue type of a tissue in a region of an HF electrode based on optical measurement signals captured by an optical capturing device; and
setting a specific HF mode for the tissue type based on the result of the optical classification.
